(19) United States
(12) Patent Application Publication
(10) Pub. No.: US 2011/0230431 A1
(43) Pub. Date: Sep. 22, 2011

(54) FOSFOMYCIN/TOBRAMYCIN COMBINATIONS FOR THE TREATMENT AND PREVENTION OF OPHTHALMIC, OTOLOGICAL AND DERMATOLOGICAL INFECTIONS

(75) Inventor: David MacLeod, Issaquah, WA (US)

(73) Assignee: Gilead Sciences, Inc., Foster City, CA (US)

(21) Appl. No.: 13/124,776

(22) PCT Filed: Oct. 16, 2009
(86) PCT No.: PCT/US09/61033

§ 371 Date: Jun. 13, 2011

Related U.S. Application Data
(60) Provisional application No. 61/107,242, filed on Oct. 21, 2008.

(51) Int. Cl. A61K 31/7036; A61P 31/04; A61P 29/00

(52) U.S. Cl. 514/40

(57) ABSTRACT

Provided are topical fosfomycin-tobramycin compositions for the treatment and/or prevention of ophthalmic, otological, and dermatologic inflammation and/or bacterial infections and methods of treating ophthalmic, otological and dermatological inflammation and/or bacterial infections.

### FIELD OF INVENTION

This invention comprises a novel physiologically compatible, topical composition of fosfomycin plus tobramycin suitable for the treatment and prevention of ophthalmic, otological and dermatological infections caused by bacteria and methods of using the composition.

### BACKGROUND OF THE INVENTION

Ophthalmic, otological, and dermatological infections have been treated with a variety of topical compositions of antibiotics including penicillins, cephalosporins, fluoroquinolones, and aminoglycosides such as amikacin, gentamicin and tobramycin. Tobramycin is commercially marketed alone and in combination with dexamethasone or loteprednol (Tobrex™, Tobradex™ and Zylet™, respectively) for the treatment and/or prevention of ophthalmic infections and has also been used for the treatment of ear infections. Tobramycin treatment is effective against common bacterial eye and ear pathogens such as Staphylococci, including S. aureus, S. epidermidis, including methicillin resistant strains; Streptococci, including S. pneumoniae, Pseudomonas aeruginosa, Escherichia coli, Klebsiella pneumoniae, Enterobacter aerogens, Proteus mirabilis, Morganella morganii, Haemophilus influenzae, H aegyptius, Acinetobacter calcoaceticus and some Neissaria species. However, tobramycin treatment has been associated with ototoxicity and increasing bacterial resistance has also been observed. Therefore, there is a need to develop compositions of tobramycin that are safer and less prone to produce resistant strains of bacteria.

Baker (WO2005/110022, which is incorporated herein by reference in its entirety), discloses aerosolized fosfomycin/tobramycin combination compositions for the treatment of bacterial respiratory infections. The rationale for the combination was to provide a new aerosol antibiotic therapy for treatment of respiratory infections that is safe, kills a broad spectrum of bacteria, and has a reduced frequency of resistance relative to monotherapies. These unique antibiotic combinations comprise a fixed (wt:wt) ratio of fosfomycin and tobramycin. The combinations are active against both gram-negative and gram-positive bacterial pathogens commonly found in respiratory infections. These combinations offer an approach to delaying development of resistance in the clinical setting because, compared to the individual component antibiotics, the combination is believed to lower the incidence of spontaneous mutation frequencies that can result in antibiotic resistance.

### SUMMARY OF THE INVENTION

It has now been discovered that combining fosfomycin with tobramycin uniquely increases the activity of tobramycin against bacteria by enhancing the uptake of tobramycin by the bacteria. As a result, the required doses of tobramycin for treating an infection are reduced in these combinations, avoiding the potential for ototoxicity that has been associated with tobramycin's use. In addition, the enhanced uptake of tobramycin with these combinations produces a rapid killing of the bacteria and diminishes the development of bacterial resistance. Therefore, the combinations of fosfomycin with tobramycin now find use beyond its previously understood pulmonary application.

The instant invention provides a method of treating ophthalmic, otological or dermatological bacterial infections by administering a therapeutically effective amount of a physiologically compatible topical composition comprising a combination of fosfomycin, or pharmaceutically acceptable salt thereof, plus tobramycin, or pharmaceutically acceptable salt thereof, to a subject in need thereof.

In another aspect, the invention provides a physiologically compatible topical composition comprising a combination of fosfomycin, or pharmaceutically acceptable salt thereof, plus tobramycin, or pharmaceutically acceptable salt thereof, suitable for the treatment of ophthalmic, otological, or dermatological infections caused by bacteria.

In another aspect, the invention provides a method of treating an ophthalmic, otological or dermatological bacterial infection and inflammation by administering a therapeutically effective amount of a physiologically compatible topical composition comprising a combination of fosfomycin, or pharmaceutically acceptable salt thereof, plus tobramycin, or pharmaceutically acceptable salt thereof, and an anti-inflammatory agent to a subject in need thereof.

In another aspect, the invention provides a physiologically compatible topical composition comprising a combination of fosfomycin, or pharmaceutically acceptable salt thereof, plus tobramycin, or pharmaceutically acceptable salt thereof, and an anti-inflammatory agent suitable for the treatment of ophthalmic, otological or dermatological inflammation and infections caused by bacteria.

In another aspect, the invention provides a method of treating an ophthalmic, otological or dermatological bacterial infection and inflammation by administering a therapeutically effective amount of a physiologically compatible topical composition comprising a combination of fosfomycin, or pharmaceutically acceptable salt thereof, plus tobramycin, or pharmaceutically acceptable salt thereof, and a corticosteroid to a subject in need thereof.

In another aspect, the invention provides a physiologically compatible topical composition comprising a combination of fosfomycin, or pharmaceutically acceptable salt thereof, plus tobramycin, or pharmaceutically acceptable salt thereof, and a corticosteroid suitable for the treatment of ophthalmic, otological or dermatological inflammation and infection caused by bacteria.

### BRIEF DESCRIPTION OF THE FIGURES

FIG. 1. FT4:1 Exhibited Concentration-dependent Killing of P. aeruginosa. “FT4:1” comprises a 4:1 ratio (wt:wt basis) of fosfomycin and tobramycin.

FIG. 2. Tobramycin Exhibited Concentration-dependent Killing of P. aeruginosa.

FIG. 3. Fosfomycin Exhibited Time-dependent Killing of P. aeruginosa.

FIG. 4. FT4:1 Exhibited Enhanced Killing of P. aeruginosa Relative to Fosfomycin and Tobramycin.

FIG. 5. FT4:1 Exhibited Rapid Inhibition of Protein Synthesis.

FIG. 6. FT4:1 Exhibited Gradual Inhibition of Cell Wall Synthesis.

FIG. 7. Fosfomycin Increases Uptake of Tobramycin in a Dose-dependent Manner.

FIG. 8. FT4: I Exhibited Enhanced Killing Relative to Fosfomycin and Tobramycin against a Clinical Isolate of CF P. aeruginosa (COR-273).

### DETAILED DESCRIPTION OF THE INVENTION

The embodiments described herein represent certain aspects of the invention and are not intended to be limiting. Additional objects, aspects and embodiments would be apparent to one skilled in the art and are intended to be encompassed by the instant invention.

In one aspect, the invention comprises a physiologically compatible topical composition for treatment of a susceptible ophthalmic, otological or dermatological bacterial infection, the method comprising a single dose combination of about 0.001 to about 0.95 mg of fosfomycin, or pharmaceutically acceptable salt thereof, and about 0.001 to about 0.95 mg of tobramycin, or a pharmaceutically acceptable salt thereof, wherein the weight ratio of fosfomycin to tobramycin is from about 5 to about 9 parts fosfomycin to about 1 part to about 5 parts tobramycin. In one embodiment of this aspect, the weight ratio of fosfomycin to tobramycin is from about 7 to about 9 parts fosfomycin to about 1 part to about 3 parts tobramycin. In a preferred embodiment of this aspect, the weight ratio of fosfomycin to tobramycin is about 8 parts fosfomycin to about 2 parts tobramycin. In another preferred embodiment of this aspect, the single dose combination of fosfomycin and tobramycin comprises about 0.1 to 0.5 percent of the composition and the weight ratio of fosfomycin to tobramycin is about 8 parts fosfomycin to about 2 parts tobramycin. In another preferred embodiment, the single dose combination comprises less than about 0.3 mg of tobramycin. In a particularly preferred embodiment, the single dose combination comprises less than about 0.15 mg of tobramycin. In another preferred embodiment, the weight ratio of fosfomycin to tobramycin is about 8 parts fosfomycin to about 2 parts tobramycin and the single dose combination comprises less than about 0.75 mg of the fosfomycin and tobramycin combination. In another embodiment of this aspect, the treatment is for a susceptible ophthalmic bacterial infection. In another embodiment of this aspect, the treatment is for a susceptible otological bacterial infection. In another embodiment of this aspect, the treatment is for a susceptible dermatological bacterial infection. In another embodiment of this aspect, the composition is an aqueous or saline solution. In another embodiment of this aspect, the composition is a gel. In another embodiment of this aspect, the composition is an ointment. In another embodiment of this aspect, the composition is a cream. In another embodiment of this aspect, the composition is a suspension. In another embodiment of this aspect, the composition is a lotion. In another embodiment of this aspect, the composition is an emulsion.

In another aspect, the invention comprises a physiologically compatible topical composition for treatment of a susceptible ophthalmic, otological or dermatological bacterial infection and inflammation, the composition comprising a single dose combination of about 0.001 to about 0.95 mg of fosfomycin, or pharmaceutically acceptable salt thereof, and about 0.001 to about 0.95 mg of tobramycin, or a pharmaceutically acceptable salt thereof, wherein the weight ratio of fosfomycin to tobramycin is from about 5 to about 9 parts fosfomycin to about 1 part to about 5 parts tobramycin, the composition further comprising about 0.001 to about 2 weight percent of at least one anti-inflammatory agent. In one embodiment of this aspect, the weight ratio of fosfomycin to tobramycin is from about 7 to about 9 parts fosfomycin to about 1 part to about 3 parts tobramycin. In a preferred embodiment of this aspect, the weight ratio of fosfomycin to tobramycin is about 8 parts fosfomycin to about 2 parts tobramycin. In another preferred embodiment of this aspect, the single dose combination of fosfomycin and tobramycin comprises about 0.1 to 0.5 percent of the composition and the weight ratio of fosfomycin to tobramycin is about 8 parts fosfomycin to about 2 parts tobramycin. In another preferred embodiment, the single dose combination comprises less than about 0.3 mg of tobramycin. In a particularly preferred embodiment, the single dose combination comprises less than about 0.15 mg of tobramycin. In another preferred embodiment, the weight ratio of fosfomycin to tobramycin is about 8 parts fosfomycin to about 2 parts tobramycin and the single dose combination comprises less than about 0.75 mg of the fosfomycin and tobramycin combination. In one embodiment of this aspect, the anti-inflammatory agent is a non-steroidal anti-inflammatory agent. In another embodiment of this aspect, the anti-inflammatory agent is at least one corticosteroid. In another embodiment of this aspect, the anti-inflammatory agent is diclofenac or ketorolac. In another embodiment of this aspect, the anti-inflammatory agent is dexamethasone or dexamethasone sodium phosphonate. In another embodiment of this aspect, the anti-inflammatory agent is fluorometholone or fluorometholone acetate. In another embodiment of this aspect, the anti-inflammatory agent is loteprednol or loteprednol etabonate. In another embodiment of this aspect, the treatment is for a susceptible ophthalmic bacterial infection and inflammation. In another embodiment of this aspect, the treatment is for a susceptible otological bacterial infection and inflammation. In another embodiment of this aspect, the treatment is for a susceptible dermatological bacterial infection and inflammation. In another embodiment of this aspect, the composition is an aqueous or saline solution. In another embodiment of this aspect, the composition is a gel. In another embodiment of this aspect, the composition is an ointment. In another embodiment of this aspect, the composition is a cream. In another embodiment of this aspect, the composition is a suspension. In another embodiment of this aspect, the composition is a lotion. In another embodiment of this aspect, the composition is an emulsion.

In another aspect, the invention comprises a physiologically compatible topical composition for a treatment of a susceptible ophthalmic, otological or dermatological bacterial infection and inflammation comprising a single dose combination of about 0.001 to about 0.95 mg of fosfomycin, or pharmaceutically acceptable salt thereof, and about 0.001 to about 0.95 mg of tobramycin, or a pharmaceutically acceptable salt thereof, wherein the weight ratio of fosfomycin to tobramycin is from about 5 to about 9 parts fosfomycin to about 1 part to about 5 parts tobramycin, the composition further comprising 0.001 to about 2 weight percent of at least one corticosteroid. In one embodiment of this aspect, the weight ratio of fosfomycin to tobramycin is from about 7 to about 9 parts fosfomycin to about 1 part to about 3 parts tobramycin. In a preferred embodiment of this aspect, the weight ratio of fosfomycin to tobramycin is about 8 parts fosfomycin to about 2 parts tobramycin. In another preferred embodiment of this aspect, the single dose combination of fosfomycin and tobramycin comprises about 0.1 to 0.5 percent of the composition and the weight ratio of fosfomycin to tobramycin is about 8 parts fosfomycin to about 2 parts tobramycin. In another preferred embodiment, the single dose combination comprises less than about 0.3 mg of tobramycin. In a particularly preferred embodiment, the single dose combination comprises less than about 0.15 mg of tobramycin. In another preferred embodiment, the weight ratio of fosfomycin to tobramycin is about 8 parts fosfomycin to about 2 parts tobramycin and the single dose combination comprises less than about 0.75 mg of the fosfomycin and tobramycin combination. In another embodiment of this aspect, the corticosteroid is dexamethasone or dexamethasone sodium phosphate. In another embodiment of this aspect, the corticosteroid is fluorometholone or fluorometholone acetate. In another embodiment of this aspect, the corticosteroid is loteprednol or loteprednol etabonate. In another embodiment of this aspect, the corticosteroid is hydrocortisone. In another embodiment of this aspect, the corticosteroid is prednisolone. In another embodiment of this aspect, the corticosteroid is fludrocortisone. In another embodiment of this aspect, the corticosteroid is triamcinolone or triamcinolone acetonide. In another embodiment of this aspect, the corticosteroid is betamethasone. In another embodiment of this aspect, the corticosteroid is beclomethasone diproprionate. In another embodiment of this aspect, the corticosteroid is methylprednisolone. In another embodiment of this aspect, the corticosteroid is fluocinolone or fluocinolone acetonide. In another embodiment of this aspect, the corticosteroid is flunisolide. In another embodiment of this aspect, the corticosteroid is fluocortin-21-butylate. In another embodiment of this aspect, the corticosteroid is flumethasone or flumetasone pivalate. In another embodiment of this aspect, the corticosteroid is budesonide. In another embodiment of this aspect, the corticosteroid is halobetasol propionate. In another embodiment of this aspect, the corticosteroid is mometasone furoate. In another embodiment of this aspect, the corticosteroid is fluticasone propionate. In another embodiment of this aspect, the corticosteroid is ciclesonide. In a preferred embodiment of this aspect, the corticosteroid is about 0.1 weight percent dexamethasone or dexamethasone sodium phosphate. In another preferred embodiment of this aspect, the corticosteroid is about 0.1 weight percent fluorometholone acetate. In another preferred embodiment of this aspect, the corticosteroid is about 0.5 weight percent loteprednol etabonate. In another embodiment of this aspect, the composition further comprises about 0.001 to about 2 weight percent each of at least two corticosteroids. In another embodiment of this aspect, the treatment is for a susceptible ophthalmic bacterial infection and inflammation. In another embodiment of this aspect, the treatment is for a susceptible otological bacterial infection and inflammation. In another embodiment of this aspect, the treatment is for a susceptible dermatological bacterial infection and inflammation. In another embodiment of this aspect, the composition is an aqueous or saline solution. In another embodiment of this aspect, the composition is a gel. In another embodiment of this aspect, the composition is an ointment. In another embodiment of this aspect, the composition is a cream. In another embodiment of this aspect, the composition is a suspension. In another embodiment of this aspect, the composition is a lotion. In another embodiment of this aspect, the composition is an emulsion.

The compositions of the instant invention are intended for the treatment of susceptible bacterial infections of the eyes (ophthalmic), ears (otological), and skin (dermatological). Inflammation is often associated with these bacterial infections, in which case the compositions may comprise an additional anti-inflammatory agent such as a non-steroidal anti-inflammatory agent or one or more corticosteroids. Non-limiting examples of susceptible bacterial infections include those caused by Staphylococci, including S. aureus, S. epidermidis, including methacillin resistant strains; Streptococci, including S. pneumoniae, Pseudomonas aeruginosa, Escherichia coli, Klebsiella pneumoniae, Enterobacter aerogens, Proteus mirabilis, Morganella morganii, Haemophilus influenzae, H. aegyptius, Acinetobacter calcoaceticus and some Neissaria species.

Non-limiting examples of corticosteroids that may be used to treat inflammation include dexamethasone, dexamethasone sodium phosphate, fluorometholone, fluorometholone acetate, loteprednol, loteprednol etabonate, hydrocortisone, prednisolone, fludrocortisones, triamcinolone, triamcinolone acetonide, betamethasone, beclomethasone diproprionate, methylprednisolone, fluocinolone, fluocinolone acetonide, flunisolide, fluocortin-21-butylate, flumethasone, flumetasone pivalate, budesonide, halobetasol propionate, mometasone furoate, fluticasone propionate, ciclesonide; and pharmaceutically acceptable salts thereof. Typically, the corticosteroids of the invention, when present, comprise about 0.001 to about 2 percent by weight of the composition; more typically about 0.1 to about 1 percent by weight of the composition.

Corticosteroids alone are often used to treat topical inflammation of the eye, ear, and skin. However, corticosteroids produce immuno-suppression that can lead to enhanced susceptibility to bacterial infection. Therefore, the fosfomycin-tobramycin compositions of the instant invention are useful for the prevention of susceptible bacterial infections when corticosteroids are administered, i.e., in patients currently being treated with a corticosteroid. Non-limiting examples of ear conditions that are treatable with the instant invention are otitis externa including complications such as ear canal stenosis, tympanic membrane perforation, auricular cellulitis and necrotizing otitis externa; otitis media with perforated tympanic membrane, particular that associated with typanostomy tubes and chronic suppurative otitis media; and other ear conditions associated with external ear infections or tympanic membrane perforation.

Non-limiting examples of eye conditions that are treatable with the instant invention are bacterial conjunctivitis and bacterial keratitis.

Non-limiting examples of skin conditions that are treatable with the instant invention are impetigo, folliculitis, furunculosis and carbunculosis. The instant invention may also be applied to wounds, cuts, insect bites and abrasions of the skin to prevent bacterial infections.

The instant invention is also useful for preventing susceptible bacterial infections when there are wounds, cuts, and abrasions to the skin, eye, or ear. The preventive properties of the instant invention are particularly useful after surgery to prevent nosocomial infections and when injuries occur in soiled working conditions or playgrounds.

In another aspect, the invention comprises a method of treating a susceptible ophthalmic, otological or dermatological bacterial infection by administering, to a subject in need thereof, a therapeutically effective amount of a physiologically compatible topical composition comprising a single dose combination of about 0.001 to about 0.95 mg of fosfomycin, or pharmaceutically acceptable salt thereof, and about 0.001 to about 0.95 mg of tobramycin, or a pharmaceutically acceptable salt thereof, wherein the weight ratio of fosfomycin to tobramycin is from about 5 to about 9 parts fosfomycin to about 1 part to about 5 parts tobramycin. In one embodiment of this aspect, the weight ratio of fosfomycin to tobramycin is from about 7 to about 9 parts fosfomycin to about 1 part to about 3 parts tobramycin. In a preferred embodiment of this aspect, the weight ratio of fosfomycin to tobramycin is about 8 parts fosfomycin to about 2 parts tobramycin. In another preferred embodiment of this aspect, the single dose combination of fosfomycin and tobramycin comprises about 0.1 to 0.5 percent of the composition and the weight ratio of fosfomycin to tobramycin is about 8 parts fosfomycin to about 2 parts tobramycin. In another preferred embodiment, the single dose combination comprises less than about 0.3 mg of tobramycin. In a particularly preferred embodiment, the single dose combination comprises less than about 0.15 mg of tobramycin. In another preferred embodiment, the weight ratio of fosfomycin to tobramycin is about 8 parts fosfomycin to about 2 parts tobramycin and the single dose combination comprises less than about 0.75 mg of the fosfomycin and tobramycin combination. In another embodiment of this aspect, the susceptible bacterial infection is an ophthalmic infection. In another embodiment of this aspect, the susceptible bacterial infection is an otological infection. In another embodiment of this aspect, the susceptible bacterial infection is a dermatological infection.

In another aspect, the invention comprises a method of treating a susceptible ophthalmic, otological or dermatological bacterial infection and inflammation by administering, to a subject in need thereof, a therapeutically effective amount of a physiological compatible topical composition comprising a single dose combination of about 0.001 to about 0.95 mg of fosfomycin, or pharmaceutically acceptable salt thereof, and about 0.001 to about 0.95 mg of tobramycin, or a pharmaceutically acceptable salt thereof, wherein the weight ratio of fosfomycin to tobramycin is from about 5 to about 9 parts fosfomycin to about 1 part to about 5 parts tobramycin, the composition further comprising about 0.001 to about 2 weight percent of at least one anti-inflammatory agent. In one embodiment of this aspect, the weight ratio of fosfomycin to tobramycin is from about 7 to about 9 parts fosfomycin to about 1 part to about 3 parts tobramycin. In a preferred embodiment of this aspect, the weight ratio of fosfomycin to tobramycin is about 8 parts fosfomycin to about 2 parts tobramycin. In another preferred embodiment of this aspect, the single dose combination of fosfomycin and tobramycin comprises about 0.1 to 0.5 percent of the composition and the weight ratio of fosfomycin to tobramycin is about 8 parts fosfomycin to about 2 parts tobramycin. In another preferred embodiment, the single dose combination comprises less than about 0.3 mg of tobramycin. In a particularly preferred embodiment, the single dose combination comprises less than about 0.15 mg of tobramycin. In another preferred embodiment, the weight ratio of fosfomycin to tobramycin is about 8 parts fosfomycin to about 2 parts tobramycin and the single dose combination comprises less than about 0.75 mg of the fosfomycin and tobramycin combination. In one embodiment of this aspect, the anti-inflammatory agent is a non-steroidal anti-inflammatory agent. In another embodiment of this aspect, the anti-inflammatory agent is a corticosteroid. In another embodiment of this aspect, the anti-inflammatory agent is diclofenac or ketorolac. In another embodiment of this aspect, the anti-inflammatory agent is dexamethasone or dexamethasone sodium phosphate. In another embodiment of this aspect, the anti-inflammatory agent is fluorometholone or fluorometholone acetate. In another embodiment of this aspect, the anti-inflammatory agent is loteprednol or loteprednol etabonate. In another embodiment of this aspect, the susceptible bacterial infection is an ophthalmic infection. In another embodiment of this aspect, the susceptible bacterial infection is an otological infection. In another embodiment of this aspect, the susceptible bacterial infection is a dermatological infection.

In another aspect, the invention comprises a method of treating a susceptible ophthalmic, otological or dermatological bacterial infection and inflammation by administering, to a subject in need thereof, a therapeutically effective amount of a physiologically compatible topical composition comprising a single dose combination of about 0.001 to about 0.95 mg of fosfomycin, or pharmaceutically acceptable salt thereof, and about 0.001 to about 0.95 mg of tobramycin, or a pharmaceutically acceptable salt thereof, wherein the weight ratio of fosfomycin to tobramycin is from about 5 to about 9 parts fosfomycin to about 1 part to about 5 parts tobramycin, the composition further comprising about 0.001 to about 2 weight percent of at least one corticosteroid. In one embodiment of this aspect, the weight ratio of fosfomycin to tobramycin is from about 7 to about 9 parts fosfomycin to about 1 part to about 3 parts tobramycin. In a preferred embodiment of this aspect, the weight ratio of fosfomycin to tobramycin is about 8 parts fosfomycin to about 2 parts tobramycin. In another preferred embodiment of this aspect, the single dose combination of fosfomycin and tobramycin comprises about 0.1 to 0.5 percent of the composition and the weight ratio of fosfomycin to tobramycin is about 8 parts fosfomycin to about 2 parts tobramycin. In another preferred embodiment, the single dose combination comprises less than about 0.3 mg of tobramycin. In a particularly preferred embodiment, the single dose combination comprises less than about 0.15 mg of tobramycin. In another preferred embodiment, the weight ratio of fosfomycin to tobramycin is about 8 parts fosfomycin to about 2 parts tobramycin and the single dose combination comprises less than about 0.75 mg of the fosfomycin and tobramycin combination. In another embodiment of this aspect, the corticosteroid is dexamethasone or dexamethasone sodium phosphate. In another embodiment of this aspect, the corticosteroid is fluorometholone or fluorometholone acetate. In another embodiment of this aspect, the corticosteroid is loteprednol or loteprednol etabonate. In another embodiment of this aspect, the corticosteroid is hydrocortisone. In another embodiment of this aspect, the corticosteroid is prednisolone. In another embodiment of this aspect, the corticosteroid is fludrocortisone. In another embodiment of this aspect, the corticosteroid is triamcinolone or triamcinolone acetonide. In another embodiment of this aspect, the corticosteroid is betamethasone. In another embodiment of this aspect, the corticosteroid is beclomethasone diproprionate. In another embodiment of this aspect, the corticosteroid is methylprednisolone. In another embodiment of this aspect, the corticosteroid is fluocinolone or fluocinolone acetonide. In another embodiment of this aspect, the corticosteroid is flunisolide. In another embodiment of this aspect, the corticosteroid is fluocortin-21-butylate. In another embodiment of this aspect, the corticosteroid is flumethasone or flumetasone pivalate. In another embodiment of this aspect, the corticosteroid is budesonide. In another embodiment of this aspect, the corticosteroid is halobetasol propionate. In another embodiment of this aspect, the corticosteroid is mometasone furoate. In another embodiment of this aspect, the corticosteroid is fluticasone propionate. In another embodiment of this aspect, the corticosteroid is ciclesonide. In a preferred embodiment of this aspect, the corticosteroid is about 0.1 weight percent dexamethasone or dexamethasone sodium phosphate. In another preferred embodiment of this aspect, the corticosteroid is about 0.1 weight percent fluorometholone acetate. In another preferred embodiment of this aspect, the corticosteroid is about 0.5 weight percent loteprednol etabonate. In another embodiment of this aspect, the composition further comprises about 0.001 to about 2 weight percent each of at least two corticosteroids. In another embodiment of this aspect, the susceptible bacterial infection is an ophthalmic infection. In another embodiment of this aspect, the susceptible bacterial infection is an otological infection. In another embodiment this aspect, the susceptible bacterial infection is a dermatological infection.

Any reference to the components of the compositions of the invention described herein also includes a reference to a physiologically acceptable salt thereof. Examples of physiologically acceptable salts of the components of the compositions of the invention include salts derived from an appropriate base, such as an alkali metal or an alkaline earth (for example, Na⁺, Li⁺, K⁺, Ca⁺² and Mg⁺²), ammonium and NX₄⁺ (wherein X is C₁-C₄ alkyl). Physiologically acceptable salts of a nitrogen atom or an amino group include salts of organic carboxylic acids such as acetic, benzoic, lactic, fumaric, tartaric, maleic, malonic, malic, isethionic, lactobionic and succinic acids; organic sulfonic acids, such as methanesulfonic, ethanesulfonic, benzenesulfonic and p-toluenesulfonic acids; amino acids lysine, arginine or glutamic acid, or a neutral group such as glycine, serine, threonine, alanine, isoleucine, or leucine; and inorganic acids, such as hydrochloric, hydrobromic, sulfuric, phosphoric and sulfamic acids. For therapeutic use, salts of active ingredients of the compounds of the invention will be physiologically acceptable, i.e. they will be salts derived from a physiologically acceptable acid or base.

### DEFINITIONS

Unless stated otherwise, the following terms and phrases as used herein are intended to have the following meanings:

When trade names are used herein, applicants intend to independently include the tradename product and the active pharmaceutical ingredient(s) or device of the tradename product.

The term “treating” or “treatment”, as used herein, unless otherwise indicated, means reversing, alleviating, inhibiting the progress of, or preventing the disorder or condition to which such term applies, or one or more symptoms of such disorder or condition.

The term “therapeutically effective amount”, as used herein, is the amount of fosfomycin and tobramycin combination or fosfomycin and tobramycin and anti-inflammatory combination present in a composition described herein, such as a physiologically acceptable composition, that is needed to provide a desired level of drug in the tissue of the eye, ear, or skin to achieve an anticipated physiological response, desired biological effect, desired anti-bacterial effect, desired anti-inflammatory effect or prevention of bacterial infection when such a composition is administered topically. The precise amount will depend upon numerous factors, for example the particular formulation, the specific activity of the composition, the delivery device employed, the physical characteristics of the composition, its intended use, as well as patient considerations such as severity of the disease state, patient cooperation, etc., and can readily be determined by one skilled in the art based upon the information provided herein.

The modifier “about” used in connection with a quantity is inclusive of the stated value and has the meaning dictated by the context (e.g., includes the degree of error associated with measurement of the particular quantity).

The term “saline”, as used herein, means an aqueous solution comprising about 0.01 to about 0.9 weight percent sodium chloride.

The term “single dose combination”, as used herein, means the combination specified, e.g., fosfomycin plus tobramycin in the amounts and ratios specified or fosfomycin plus tobramycin plus anti-inflammatory agent in the amounts and ratios specified, that is administered as a single dose. In the case of liquid compositions, the single dose would typically be one or two drops. The components of the single dose combination may be premixed or combined just prior to administration.

### Composition Formulations

The physiologically compatible topical compositions of the instant invention include solutions, sprays, lotions, gels, ointments, creams, powders, dusting powder sprays, pastes, suspensions, emulsions, and foams comprising the fosfomycin and tobramycin in combination. These compositions may further comprise an anti-inflammatory agent such as, but not limited to, a non-steroidal anti-inflammatory agent or a corticosteroid. The compounds of the composition may be in dissolved or suspended form.

The fosfomycin and tobramycin compositions or fosfomycin and tobramycin compositions further comprising an anti-inflammatory agent can also be applied topically in the form of ointments, creams pastes, gels, dusting powders, plasters, spray plasters, occlusive dressings, compresses and controlled release systems.

Ointments contain, as the base, hydrocarbon gels, lipogels, absorption bases, water-in-oil ointment bases, mixed emulsions or polyethylene glycols.

Creams contain oil-in-water bases.

Pastes contain, in addition to an ointment or cream base, high amounts of pulverulent constituents, such as zinc oxide, talc, starch or titanium dioxide.

Gels contain solvents, such as water, ethanol, isopropanol or propylene glycol, and are prepared using gelling agents, such as cellulose ethers, alginates, polyacrylates, bentonite, gelatin, tragacanth, polyvinylpyrrolidone or polyvinyl alcohol.

Dusting powders contain pulverulent additives, such as starch stearate, silicon dioxide, clay, magnesium carbonate, talc, cellulose, zinc oxide and lactose.

Stabilizers, antioxidants, preservatives, humectants, regreasing agents, solvents or auxiliaries can be added to all the compositions to improve the penetration and efficacy of the active ingredients of the composition.

Non-limiting examples of agents which improve penetration are propylene glycol, polyethylene glycol, dimethylsulphoxide, deccylmethylsulphoxide, atones, N-methylpyrrolidone, diethyltoluamide, ethanol, isopropyl myristate, isopropyl palmmitate, oleic acid and its esters, medium-chain triglycerides, dimethyl isosorbitol, 2-octyldodecanol, branched fatty acids, benzyl alcohol, urea, salicylates and surfactants.

Spreading oils can also be added to the liquid form of the compositions of the invention for better distribution on surfaces, particularly for application to the skin. Many of these spreading oils are known in the cosmetic arts. Non-limiting examples of spreading oils include silicone oil of varying viscosity, fatty acid esters, triglycerides, fatty alcohols, and fatty acids, such as oleic acid. Particularly suitable spreading oils include isopropyl myristate, isopropyl palmitate, caprylic/capric acid ester of saturated fatty alcohols of C₁₂-C₁₈ chain length and waxy fatty acid esters.

The compositions of the instant invention are to be administered topically to the eye, ear, or skin. For administration to the eye or ear, the dosage range is 0.001 to 1.9 mg/per eye or ear; wherein the cited mass represents the sum of the weight of fosfomycin and tobramycin. The compositions of the instant invention can be administered as solutions, suspensions, or emulsions (dispersions) in a suitable ophthalmic or otic vehicle. While the precise dosing regimen will be determined by a physician, the solution, suspension or emulsion of the composition is typically applied by placing one or two drops in each eye for a single treatment (dose). While the volume of a drop may vary according to solution characteristics, such as viscosity and density, and dropper configuration, unless otherwise stated, the volume of a drop is about 0.05 mL. The treatment may be repeated one to 24 times a day. In one aspect, the instant topical compositions comprise about 0.01 to about 2 percent by combined weight to volume solutions of fosfomycin and tobramycin in water at a pH of about 4.5 to about 8.0. In another aspect, the topical compositions comprise about 0.01 to about 2 percent by combined weight to weight solutions or suspensions of fosfomycin and tobramycin in an ointment formulation. Other ingredients which may be desirable to use in either of the compositions include preservatives, co-solvents, surfactants and viscosity enhancing agents.

Because topical products are typically packaged in multidose form, preservatives are usually added to prevent microbial contamination during use. Non-limiting examples of suitable preservatives include benzalkonium chloride, thimerosal, chlorobutanol, methyl paraben, propyl paraben, phenylethyl alcohol, edentate disodium sorbic acid, Onamer M, or other agents known to those skilled in the art. Typically such preservatives are employed at a level of from about 0.001% to about 1.0% by weight.

The solubility of the components comprising the present composition may be enhanced by a surfactant or other appropriate co-solvent in the composition. Non-limiting examples of co-solvents and surfactants include polysorbate 20, 60, and 80, Pluronic F-68, F-84 and P-103, cyclodextrin, tyloxapol, TWEEN 80, or other agents known to those skilled in the art. Typically such co-solvents are employed at a level of from about 0.01% to about 2% by weight.

Increasing the viscosity of a topical composition above that of simple aqueous solutions may be desirable to increase absorption of the active components, to decrease variability in dispensing the formulation, to decrease physical separation of the components of a suspension or emulsion of the composition and/or to otherwise improve the topical composition. Non-limiting examples of viscosity enhancing agents include polyvinyl alcohol, polyvinyl pyrrolidone, methyl cellulose, hydroxyl propyl methylcellulose, hydroxyethyl cellulose, carboxymethyl cellulose, hydroxypropyl cellulose, povidone or other agents known to those skilled in the art. Such agents are typically employed at a level of from about 0.01% to about 2.0% by weight.

Most of the excipients, preservatives, co-solvents, surfactants and viscosity enhancing agents are described in detail in, e.g., Howard C. Ansel et al., Pharmaceutical Dosage Forms and Drug Delivery Systems, (7^{th} Ed. 1999); Alfonso R. Gennaro et al., Remington: The Science and Practice of Pharmacy, (19^{th} and 20^{th} Ed. 1995 and 2000, respectively); and A. Kibbe, Handbook of Pharmaceutical Excipients, (3^{rd} Ed. 2000) each of which is incorporated by reference in their entirety.

### EXAMPLES

The following are, non-limiting, representative pharmaceutical compositions of the instant invention for ophthalmic, otic or dermal application to treat or prevent infections by susceptible bacteria or to treat or prevent inflammation and infections by susceptible bacteria. The pharmaceutical composition examples are single dose examples which could be scaled to larger quantities by one skilled in the art. The preparation of these dosage forms are known to those skilled in the art as discussed in the references cited above and incorporated by reference. QS, unless otherwise stated, means adding a quantity sufficient to achieve a stated function; for example, to bring a solution or suspension to a desired volume or weight or adjust pH to a desired value.

### Example 1

Anti-infective Solution

| | | | |
|---|---|---|---|
| Fosfomycin, USP | 0.24 | mg | 0.24% |
| Tobramycin, USP | 0.06 | mg | 0.06% |
| Benzalkonium chloride | 0.0001 | mL | 0.1% |
| Solution (10%), NF | | | |
| Edetate disodium, USP | 0.01 | mg | 0.01% |
| Sodium chloride, USP | 0.30 | mg | 0.3% |
| Sodium sulfate, USP | 1.2 | mg | 1.2% |
| Tyloxapol, USP | 0.05 | mg | 0.05% |
| Hydroxyethylcellulose | 0.25 | mg | 0.25% |

| | | |
|---|---|---|
| Sulfuric acid and/or sodium | QS for pH | |
| hydroxide, NF | Adjustment to 5.5 | |
| Purified Water, USP | QS to 0.1 mL | QS to 100% |

### Example 2

Anti-infective Ointment

| | | | |
|---|---|---|---|
| Fosfomycin, USP micronized | 0.24 | mg | 0.24% |
| Tobramycin, USP micronized | 0.06 | mg | 0.06% |
| Chlorobutanol, Anhydrous, NF | 0.5 | mg | 0.5% |
| Mineral Oil, USP | 5 | mg | 5% |

| | | |
|---|---|---|
| White Petrolatum, USP | QS to 0.1 g | QS to 100% |

### Example 3

Anti-infective and Anti-inflammatory Suspension

| | | | |
|---|---|---|---|
| Fosfomycin, USP | 0.24 | mg | 0.24% |
| Tobramycin, USP | 0.06 | mg | 0.06% |
| Dexamethasone, Micronized, USP | 0.1 | mg | 0.1% |
| Benzalkonium chloride | 0.0001 | mL | 0.1% |
| Solution (10%), NF | | | |
| Edetate disodium, USP | 0.01 | mg | 0.01% |
| Sodium chloride, USP | 0.30 | mg | 0.3% |
| Sodium sulfate, USP | 1.2 | mg | 1.2% |
| Tyloxapol, USP | 0.05 | mg | 0.05% |
| Hydroxyethylcellulose | 0.25 | mg | 0.25% |

| | | |
|---|---|---|
| Sulfuric acid and/or sodium | QS for pH | |
| hydroxide, NF | Adjustment to 5.5 | |
| Purified Water, USP | QS to 0.1 mL | QS to 100% |

### Example 4

Anti-infective and Anti-inflammatory Suspension

| | | | |
|---|---|---|---|
| Fosfomycin, USP | 0.24 | mg | 0.24% |
| Tobramycin, USP | 0.06 | mg | 0.06% |
| Loteprednol etabonate | 0.5 | mg | 0.5% |
| Micronized, USP | | | |
| Prednisolone Micronized, USP | 0.1 | mg | 0.1% |
| Benzalkonium chloride | 0.0005 | mL | 0.5% |
| Solution (10%), NF | | | |
| Edetate disodium, USP | 0.01 | mg | 0.01% |
| ε-Aminocaproic acid | 0.1 | mg | 0.1% |
| Sodium sulfate, USP | 1.2 | mg | 1.2% |
| Tyloxapol, USP | 0.03 | mg | 0.03% |
| Polyvinylpyrrolidone | 0.6 | mg | 0.6% |
| (intrinsic viscosity = 30 | | | |

| | | |
|---|---|---|
| Hydrochloric acid | QS for pH | |
| | Adjustment to 5.5 | |
| Purified Water, USP | QS to 0.1 mL | QS to 100% |

### Example 5

Anti-infective and Anti-inflammatory Ointment

| | | | |
|---|---|---|---|
| Fosfomycin, USP Micronized | 0.24 | mg | 0.24% |
| Tobramycin, USP Micronized | 0.06 | mg | 0.06% |
| Dexamethasone, Micronized, USP | 0.1 | mg | 0.1% |
| Chlorobutanol, Anhydrous, NF | 0.5 | mg | 0.5% |
| Mineral Oil, USP | 5 | mg | 5% |

| | | |
|---|---|---|
| White Petrolatum, USP | QS to 0.1 g | QS to 100% |

### Example 6

Anti-infective and Anti-inflammatory Ointment

| | | | |
|---|---|---|---|
| Fosfomycin, USP Micronized | 0.24 | mg | 0.24% |
| Tobramycin, USP Micronized | 0.06 | mg | 0.06% |
| Loteprednol etabonate Micronized, USP | 0.2 | mg | 0.2% |
| Prednisone Micronized, USP | 0.2 | mg | 0.2% |
| Liquid paraffin | 10 | mg | 10% |

| | | |
|---|---|---|
| White soft paraffin USP | QS to 0.1 g | QS to 100% |

### Example 7

Anti-infective and Anti-inflammatory Cream

| | | | |
|---|---|---|---|
| Fosfomycin, USP micronized | 0.24 | mg | 0.24% |
| Tobramycin, USP micronized | 0.06 | mg | 0.06% |
| Dexamethasone, Micronized, USP | 0.1 | mg | 0.1% |
| Stearic acid | 14 | mg | 14% |
| Cetyl alcohol | 1 | mg | 1% |
| Isopropyl palmitate | 1 | mg | 1% |
| Methylparaben | 0.1 | mg | 0.1% |
| Propylparaben | 0.05 | mg | 0.05% |
| Sorbitan monostearate | 2 | mg | 2% |
| Sorbitol solution(70%) | 3 | mg | 3% |
| Polysorbate 60 | 1.5 | mg | 1.5% |

| | | |
|---|---|---|
| Purified Water, USP | QS to 0.1 g | QS to 100% |

### Example 8

Anti-infective and Anti-inflammatory Melting Dosage Form

| | | | |
|---|---|---|---|
| Fosfomycin, USP micronized | 0.24 | mg | 0.24% |
| Tobramycin, USP micronized | 0.06 | mg | 0.06% |
| Hydrocortisone Micronized, USP | 1.5 | mg | 1.5% |
| Cocoa Butter | 50 | mg | 50% |

| | | |
|---|---|---|
| Castor Oil | QS to 0.1 g | QS to 100% |

To promote long-term stability, one or more components of the composition may be packaged separately from the other components and subsequently added to the formulation just prior to use. For example, a solution of tobramycin and other excipients could be packaged in a bottle and the fosfomycin packaged separately in a blister pack wherein the blister pack would be opened and the fosfomycin contained therein added to the tobramycin solution just prior to use.

### Fosfomycin/Tobramycin Composition Mechanism

The purpose of this study was to address the mechanism of action hypothesis for a 4:1 fosfomycin:tobramycin, wt:wt, combination: “fosfomycin enhances the uptake of tobramycin into Pseudomonas aeruginosa, thereby increasing inhibition of protein synthesis and ultimately bacterial killing”.

### Materials and Methods

### Bacterial Strains

P. aeruginosa strains isolated from patients with cystic fibrosis (CF) were obtained from Children's Hospital and Regional Medical Center (Seattle, Wash.). Clinical isolates of Escherichia coli and Staphylococcus aureus were obtained from The Jones Group Laboratories (JMI; North Liberty, Iowa) and The Clinical Microbiology Institute (CMI; Wilsonville, Oreg.). P. aeruginosa 27853, S. aureus 29213, and E. coli 25922 served as quality control strains¹ and were obtained from The America Type Culture Collection (ATCC; Manassas, Va.). P. aeruginosa ATCC 27853, a fosfomycin and tobramycin susceptible strain, was used in macromolecular biosynthesis and tobramycin uptake experiments. Stock cultures were maintained at −80° C. in Cation-Adjusted Mueller-Hinton Broth (CAMHB) (Remel; Lenexa, Kans.) supplemented with 20% glycerol (VWR: West Chester, Pa.). Cultures for routine use were grown on tryptic soy agar plates+5% sheeps blood (PML Microbiologicals Inc.; Wilsonville, Oreg.) and stored at 4° C.

### Antibiotics

Stock solutions of fosfomycin disodium (Lot No. 023K0834, Sigma-Aldrich; St. Louis, Mo.) and tobramycin sulfate (Lot No. 124K0948; Sigma-Aldrich) were prepared in sterile deionized water and adjusted to account for potency according to Clinical and Laboratory Standards Institute guidelines (CLSI; formerly National Committee for Clinical Laboratory Standards).¹ “FT4:1” comprises a 4:1 ratio (wt:wt basis) of fosfomycin and tobramycin. Glucose-6-phosphate (Sigma-Aldrich) was added to the media at a final concentration of 25 μg/mL for all evaluations of fosfomycin and FT4:1.¹

### Reversed-Phase High Performance Liquid Chromatography/Mass Spectometry (HPLC/MS)

Stock solutions of fosfomycin (6.11 mg/mL) and tobramycin (4.22 mg/mL) were prepared in phosphate-buffered saline (PBS, pH 7.4). FT4:1 (512 μg/mL) was prepared from the stock solutions and incubated for 24 h at room temperature, 37° C. or 89° C. An Agilent 1100 Series HPLC system equipped with an LC/MSD Ion Trap Mass Spectrometer and ChemStation data acquisition/data analysis software (Agilent Technologies; Santa Clara, Calif.) was used to detect potential chemical adducts of fosfomycin and tobramycin. Peak separation was effected using a Symmetryshield RP18 analytical column, 4.6 mm i.d.×150 mm length, with 3.5 μm packing (Waters Corporation; Milford, Mass.). The samples were eluted with 5% glacial acetic acid and 0.25% pentafluoropropionic acid (PFPA) in water as mobile phase A, and 5% glacial acetic acid and 0.25% PFPA in acetonitrile as mobile phase B. An elution gradient was applied from 0% to 34% mobile phase B over 25 minutes. Peaks were eluted directly from the column into the electrospray ionization source of the ion trap mass spectrometer. Ionization was in positive mode, using nitrogen as a drying gas at 10 L/min and 350 ° C. Mass spectra were acquired over a range of 150 to 1300 m/z.

### Minimal Inhibitory Concentration (MIC)

MICs were determined by the agar plate dilution method according to CLSI guidelines (National Committee for Clinical Laboratory Standards. M7-A6. 6^{th} ed. Villanova (Pa.): NCCLS, 2003; M100-S14. Villanova (Pa.): NCCLS, 2004). The MIC was defined as the lowest concentration of antibiotic that prevented visible growth after incubation at 37° C. for 18-24 hours.

### Time-Kill Experiments

Time-kill experiments were performed according to a modified CLSI method (National Committee for Clinical Laboratory Standards. M-26A. Villanova (Pa.): NCCLS, 1999). Antibiotics were evaluated alone and in combination at multiples of the MIC in CAMHB containing 2% porcine gastric mucin (Sigma-Aldrich). Bacterial cultures and antibiotic(s) were incubated at 37° C. in a shaking water bath (200 rpm) and killing activity assessed at 0, 1, 2, 4, 6 and 24 h. Antibiotics that reduced the original inoculum by ≧3-Log₁₀ were considered bactericidal. Antibiotics that reduced the original inoculum by ≦2-Log₁₀ were considered bacteriostatic.

### Spontaneous Mutation Frequencies

Development of resistance after a single exposure to antibiotic was determined for both clinical and reference isolates of P. aeruginosa, S. aureus, and E. coli. Late Log-phase cultures were centrifuged at 1,400×g at room temperature for 20 min and the cell pellets resuspended in CAMHB. Approximately 10⁹-10¹⁰ CFU were spread onto Mueller-Hinton agar (BBL; Sparks, Md.) plates containing 4×, 8×, or 16× the MIC of antibiotic. The culture plates were incubated at 37° C. for 48 h and the number of colonies on each plate was enumerated manually. The spontaneous mutation frequency (SMF) resulting in antibiotic resistance was calculated by dividing the number of bacteria growing at the defined antibiotic concentration by the number of bacteria in the inoculum (Martinex, J L, Antimicrob Agents Chemother 2000; 44 (1):1771-1777).

### Macromolecular Biosynthesis

The effects of FT4:1, fosfomycin and tobramycin on protein and cell wall biosynthesis were determined by measuring the incorporation of tritiated (³H) amino acids (³H-aa) (GE Healthcare Bio-Sciences Corp.; Picataway, N.J.) and N-acetyl-D-Glucosamine (³H-NAG) (GE Healthcare Bio-Sciences), respectively (Baum, E Z, Antimicrob Agents Chernother 2001; 45 (11): 3182-3188). A single colony of P. aeruginosa ATCC 27853 was inoculated into 10 mL of CAMHB and incubated for 16 h, at 37° C., 200 rpm in a shaking water bath. The culture was diluted 1:1000 in 50 mL CAMHB+2% mucin in a 125 mL Erlenmeyer flask and incubated at 37° C., 200 rpm for 1.5 h. Two milliliters of early log phase cultures (˜2×10⁷ CFU/mL) were pulsed with 10 μCi of ³H-aa (1.93 GBq/milliatom carbon) or 10 μCi of ³H-NAG (296 GBq/mmol) for 1 h at 37° C., 200 rpm. Non-radioactive FT4:1, fosfomycin, or tobramycin were then added to cultures and incubated as described above for up to an additional 4 h.

At various time points, 100 μl aliquots (triplicate) of culture were removed and added to 100 μl of 20% TCA (VWR) in 96-well flat bottom trays (VWR). Plates were incubated on crushed ice for 60 min to precipitate the incorporated ³H-precursors. Samples were harvested onto glass fibre filters (GFC) (PerkinElmer; Waltham, Mass.). Filters were washed two times with 35 mL of normal saline to remove unincorporated isotope followed by one wash with 35 mL of 90% ETOH (VWR). Filters were dried under a lamp for 1 h to reduce remaining moisture and then sealed in bags containing 5 mL of scintillation cocktail (Betaplate Scint; PerkinElmer). Counts per minute (CPM) were determined using a Wallac MicroBeta Trilux (PerkinElmer).

Mean CPM±standard deviation (SD) were determined for each treatment group. Background counts consisting of a media-only sample were subtracted from the no-drug and antibiotic treatment groups. The percent inhibition of incorporation was determined relative to the no-drug controls. The activities of the three drugs were compared by calculating the time to 50% inhibition (T_{1/2}), and statistical differences at each time point were evaluated by the Analysis of Variance (ANOVA; GraphPad Prisma software package 3.03; GraphPad Software, Inc., San Diego, Calif.). Each curve represents 4 replicate experiments, each time point performed in triplicate.

### Tobramycin Uptake Studies

The effects of fosfomycin uptake on tobramycin uptake were determined by measuring uptake of ³H-tobramycin. A single colony of P. aeruginosa ATCC 27853 was inoculated into 5 mL nutrient broth (NB) (Difco & BBL; Sparks, Md.) and grown overnight at 37° C. with shaking (250 rpm). The overnight culture was diluted in NB to an OD₆₂₅ of 0.013 and cultured at 37° C. with shaking (250 rpm) until it reached an 0D₆₂₅ of ˜0.5. Cells were harvested by centrifugation (6000×g, room temperature, 5 min), washed once in NB and resuspended in pre-warmed NB to an OD₆₂₅ of 0.25. Unlabeled fosfomycin was then added at the appropriate concentration (0, 0.05, 0.1, 1, 10, and 100 μg/mL) and the cultures incubated for 3 min at 37° C. with shaking (250 rpm). ³H-tobramycin (540 mCi/mmol, Moravek Biochemicals; Brea, Calif.) (2.3 μg/mL) was added to each tube and the cultures were incubated at 37° C. with shaking (250 rpm) for an additional 2 min. Five milliliter volumes were filtered through 0.45 μm nitrocellulose membrane filters (Whatman Inc., Florham Park, N.J.), pre-soaked with 410 mM MgCl₂ (VWR). Filters were dried overnight, saturated with 3 mL Betaplate Scint, and the ³H associated with each filter was determined with a MicroBeta scintillation counter. Data were expressed in CPM and represent the mean±SD of four independent experiments. Statistical differences were evaluated by the Student's t-test.

Results

### Reversed-Phase High Performance Liquid Chromatography/Mass Spectrometry (HPLC/MS)

Based on the number of primary amines on tobramycin, formation of five possible adducts were feasible. However, no chemical adducts of fosfomycin and tobramycin were detected by HPLC/MS after incubation of FT4:1 at room temperature or 37° C. for 24 hours. This suggests the enhanced antibacterial activities were not due to new chemical entity.

### Time-Kill Experiments

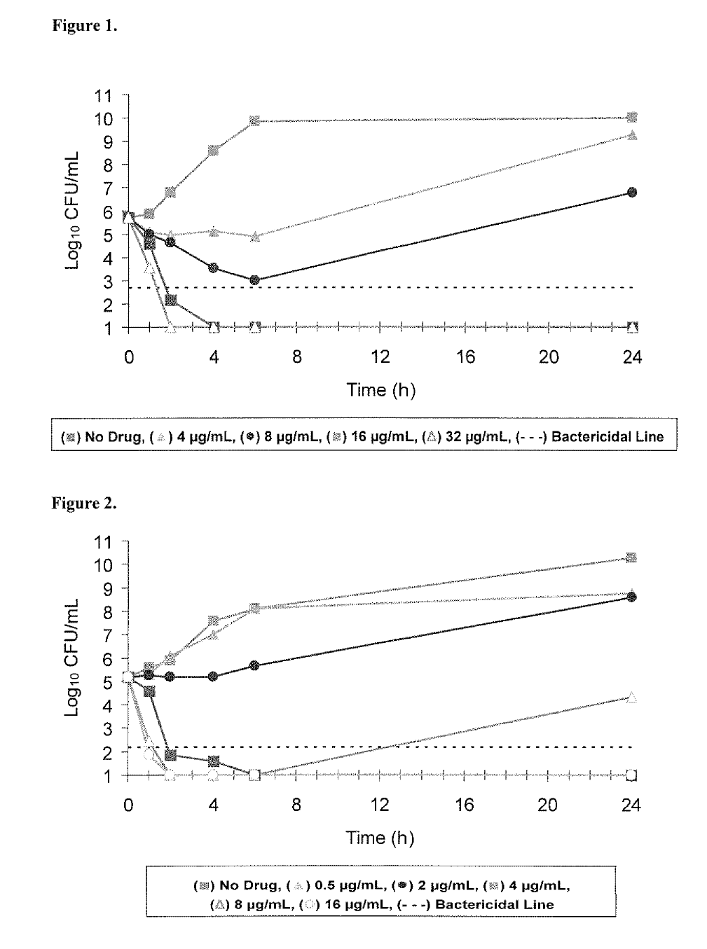

Time-kill experiments demonstrated that the more active component of FT4:1 was tobramycin; both FT4:1 (FIG. 1) and tobramycin (FIG. 2) were rapidly bactericidal (≧3-Log₁₀ killing) and exhibited concentration-dependent killing. Increasing the concentrations of FT4:1 and tobramycin significantly increased both the rate and extent of bacterial killing. For tobramycin, increasing the concentration from 2 μg/mL to 4 μg/mL (2-fold) resulted in >4-Log₁₀ of bacterial killing (FIG. 2).

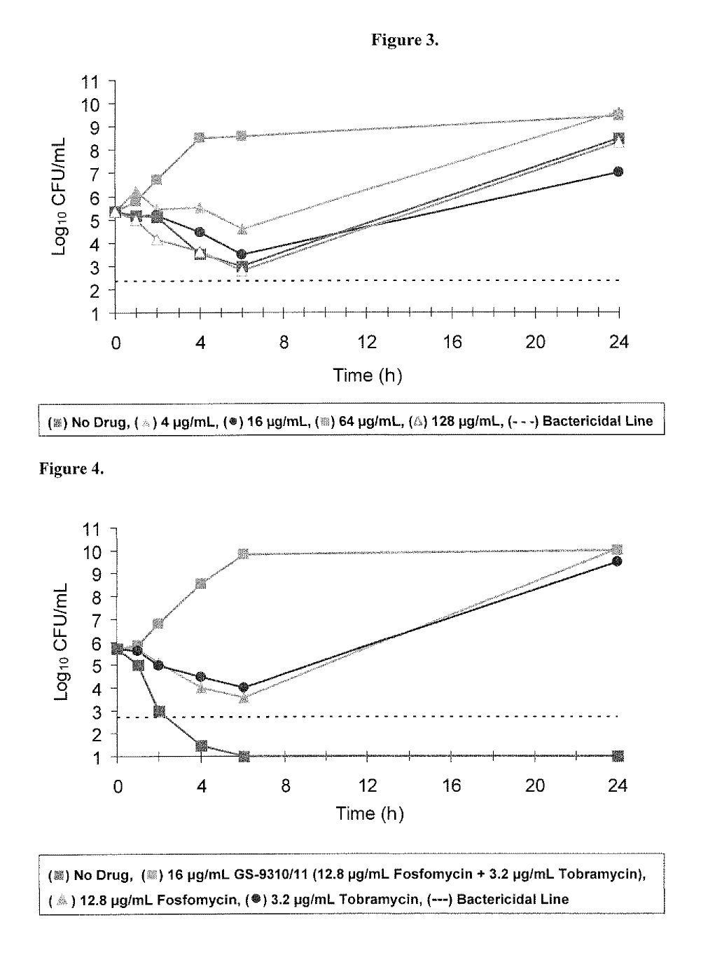

By comparison, fosfomycin was bacteriostatic (≦2-Log₁₀ killing) and killed in a time-dependent fashion (FIG. 3). Increasing the fosfomycin concentration (≦32×MIC) did not produce a significant increase in the rate or degree of bacterial killing.

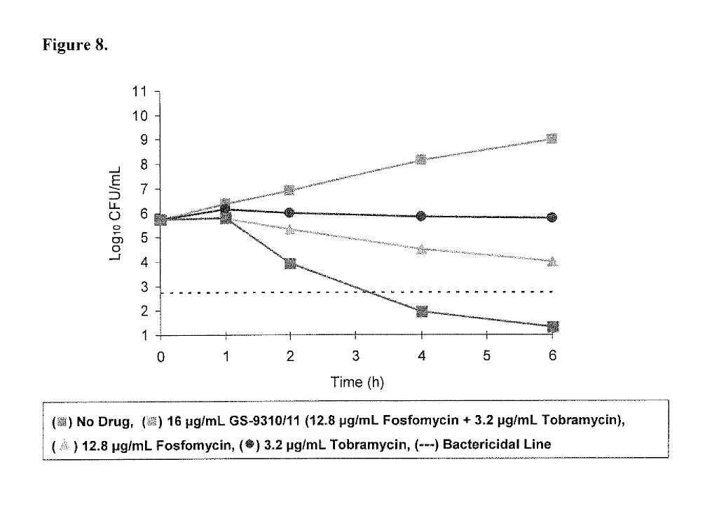

FIG. 4 shows the activity of FT4:1 relative to its component weights of fosfomycin and tobramycin against P. aeruginosa ATCC 27853 in the presence of mucin. At 4×MIC (16 μg/mL), the killing activity of FT4:1 was superior relative to its components fosfomycin (12.8 μg/mL) and tobramycin (3.2 μg/mL). FT4:1 rapidly reached bactericidal killing (1-2 h), while tobramycin and fosfomycin alone exhibited bacteriostatic killing. Additionally, FT4:1 remained bactericidal at 24 h, while both tobramycin and fosfomycin exhibited re-growth of the bacterial culture. The time-kill experiments suggest that the enhanced antibacterial activity of FT4:1 results from facilitated tobramycin uptake. These studies also demonstrated that exposure to very small increases in tobramycin concentration (2-fold) resulted in large increases (3-Log₁₀ CFU/mL) in bacterial killing. Similar activities were observed with a CF clinical isolate of P. aeruginosa (COR-273; FIG. 8).

### Spontaneous Mutation Frequencies

Tables 1-3 show the frequencies of spontaneous single-step mutation leading to antibiotic resistance. The spontaneous mutation frequencies for FT4:1 did not decrease as a function of the multiples of the MIC as noted with tobramycin and fosfomycin, suggesting that treatment-emergent resistance to FT4:1 may be less problematic than to the individual components of the combination. Against the five S. aureus strains, FT4:1 had the lowest mutation frequencies followed by tobramycin and fosfomycin (Table 1). At 4×MIC, FT4:1 had a mutation frequency 100- to 1000-fold less than tobramycin and 1-100 million-fold less than fosfomycin. At 8× and 16×MIC, FT4:1 and tobramycin had comparable mutation frequencies. Against P. aeruginosa, FT4:1 was superior to tobramycin, but the differences were only 10- to 100-fold (Table 2). All three antibiotics had mutation frequencies within one order of magnitude of each other against E. coli (Table 3). Fosfomycin had the highest mutation frequency for P. aeruginosa followed by E. coli and S. aureus.

TABLE 1
S. aureus Spontaneous Mutation Frequencies Resulting
in Development of Antibiotic Resistance

| | | MIC | | | |
|---|---|---|---|---|---|
| Organ- | | (μg/ | Frequency of Resistance | | |
| ism | Drug | mL) | 4× MIC | 8× MIC | 16× MIC |
| C051 | FT4:1 | 1 | <1.8 × 10⁻¹⁰ | <1.8 × 10⁻¹⁰ | <2.0 × 10⁻¹⁰ |
| | Tobramycin | 0.25 | 3.5 × 10⁻⁶ | 1.5 × 10⁻⁸ | <2.0 × 10⁻¹⁰ |
| | Fosfomycin | 1 | 3.0 × 10⁻⁵ | 3.6 × 10⁻⁷ | 8.4 × 10⁻⁶ |
| C053 | FT4:1 | 1 | <1.8 × 10⁻¹⁰ | <1.8 × 10⁻¹⁰ | <2.3 × 10⁻¹⁰ |
| | Tobramycin | 0.25 | 2.0 × 10⁻⁷ | <1.8 × 10^{-!0} | <2.3 × 10⁻¹⁰ |
| | Fosfomycin | 1 | 7.7 × 10⁻³ | 1.7 × 10⁻⁷ | 4.3 × 10⁻⁶ |
| C055 | FT4:1 | 2 | <4.3 × 10⁻⁹ | <4.3 × 10⁻⁹ | <2.3 × 10⁻¹⁰ |
| | Tobramycin | 0.25 | 3.8 × 10⁻⁷ | <4.3 × 10⁻⁹ | <2.3 × 10⁻¹⁰ |
| | Fosfomycin | 8 | 2.5 × 10⁻⁶ | <4.3 × 10⁻⁹ | 8.2 × 10⁻⁶ |
| C057 | FT4:1 | 2 | 1.0 × 10⁻⁹ | <1.4 × 10⁻¹⁰ | <3.2 × 10⁻¹⁰ |
| | Tobramycin | 0.5 | 1.1 × 10⁻⁶ | <1.4 × 10⁻¹⁰ | <3.2 × 10⁻¹⁰ |
| | Fosfomycin | 2 | 2.1 × 10⁻⁵ | 1.1 × 10⁻⁷ | 2.0 × 10⁻⁷ |
| ATCC | FT4:1 | 2 | <3.1 × 10⁻¹⁰ | <3.1 × 10⁻¹⁰ | <1.8 × 10⁻¹⁰ |
| 29213 | Tobramycin | 0.25 | 1.6 × 10⁻⁷ | 3.9 × 10⁻⁸ | <1.8 × 10⁻¹⁰ |
| | Fosfomycin | 2 | 2.6 × 10⁻⁸ | 5.0 × 10⁻⁸ | <1.8 × 10⁻¹⁰ |

TABLE 2
P. aeruginosa Spontaneous Mutation Frequencies
Resulting in Development of Antibiotic Resistance

| | | MIC | | | |
|---|---|---|---|---|---|
| Organ- | | (μg/ | Frequency of Resistance | | |
| ism | Drug | mL) | 4× MIC | 8× MIC | 16× MIC |
| C002 | FT4:1 | 4 | 5.0 × 10⁻⁶ | 3.0 × 10⁻⁷ | 4.6 × 10⁻⁸ |
| | Tobramycin | 0.5 | 1.1 × 10⁻⁵ | 1.1 × 10⁻⁷ | 4.4 × 10⁻⁸ |
| | Fosfomycin | 4 | 6.5 × 10⁻³ | 1.1 × 10⁻⁴ | 4.6 × 10⁻⁴ |
| C003 | FT4:1 | 4 | 1.1 × 10⁻⁶ | 5.1 × 10⁻⁶ | 6.0 × 10⁻⁸ |
| | Tobramycin | 0.5 | 4.2 × 10⁻⁵ | 1.8 × 10⁻⁷ | 7.2 × 10⁻⁸ |
| | Fosfomycin | 16 | 1.1 × 10⁻⁶ | 9.0 × 10⁻⁵ | 1.3 × 10⁻⁴ |
| C013 | FT4:1 | 4 | 1.2 × 10⁻⁷ | 1.3 × 10⁻⁷ | 6.3 × 10⁻⁷ |
| | Tobramycin | 0.5 | 1.4 × 10⁻⁶ | 3.7 × 10⁻⁶ | 8.2 × 10⁻⁷ |
| | Fosfomycin | 32 | 9.2 × 10⁻³ | 1.3 × 10⁻⁵ | 1.3 × 10⁻⁷ |
| C014 | FT4:1 | 4 | 3.4 × 10⁻⁶ | 1.6 × 10⁻⁷ | 5.4 × 10⁻⁷ |
| | Tobramycin | 0.5 | 1.3 × 10⁻⁶ | 1.1 × 10⁻⁷ | 7.9 × 10⁻⁷ |
| | Fosfomycin | 8 | 1.4 × 10⁻⁴ | 1.3 × 10⁻⁶ | 1.8 × 10⁻⁶ |
| ATCC | FT4:1 | 4 | 4.6 × 10⁻⁷ | 5.1 × 10⁻⁷ | 4.1 × 10⁻⁸ |
| 27853 | Tobramycin | 0.5 | 3.0 × 10⁻⁵ | 2.2 × 10⁻⁷ | 1.6 × 10⁻⁸ |
| | Fosfomycin | 4 | 7.2 × 10⁻⁴ | 1.1 × 10⁻⁵ | 7.2 × 10⁻⁵ |

TABLE 3
E. coli Spontaneous Mutation Frequencies Resulting
in Development of Antibiotic Resistance

| | | MIC | | | |
|---|---|---|---|---|---|
| Organ- | | (μg/ | Frequency of Resistance | | |
| ism | Drug | mL) | 4× MIC | 8× MIC | 16× MIC |
| C032 | FT4:1 | 0.5 | 1.4 × 10⁻⁶ | 1.7 × 10⁻⁷ | 2.9 × 10⁻⁷ |
| | Tobramycin | 0.5 | 1.3 × 10⁻⁶ | 5.5 × 10⁻⁶ | 6.2 × 10⁻⁷ |
| | Fosfomycin | 1 | 4.4 × 10⁻⁵ | 7.8 × 10⁻⁵ | 2.1 × 10⁻⁶ |
| C036 | FT4:1 | 0.5 | 2.2 × 10⁻⁶ | 1.6 × 10⁻⁷ | 3.1 × 10⁻⁷ |
| | Tobramycin | 0.5 | 4.4 × 10⁻⁶ | 4.7 × 10⁻⁷ | 1.9 × 10⁻⁹ |
| | Fosfomycin | 2 | 1.1 × 10⁻⁶ | 2.3 × 10⁻⁶ | 3.4 × 10⁻⁶ |
| C037 | FT4:1 | 1 | 1.4 × 10⁻⁶ | 4.3 × 10⁻⁶ | 7.3 × 10⁻⁶ |
| | Tobramycin | 4 | 1.0 × 10⁻⁶ | 4.4 × 10⁻⁶ | 1.1 × 10⁻⁷ |
| | Fosfomycin | 1 | 1.9 × 10⁻⁶ | 2.9 × 10⁻⁶ | 8.6 × 10⁻⁶ |
| C294 | FT4:1 | 0.5 | 3.7 × 10⁻⁶ | 5.9 × 10⁻⁶ | 6.0 × 10⁻⁷ |
| | Tobramycin | 0.5 | 6.6 × 10⁻⁵ | 1.3 × 10⁻⁶ | <7.2 × 10⁻⁹ |
| | Fosfomycin | 0.5 | 7.9 × 10⁻⁵ | 5.4 × 10⁻⁵ | 6.3 × 10⁻⁵ |
| ATCC | FT4:1 | 1 | 6.0 × 10⁻⁶ | 1.3 × 10⁻⁸ | 2.0 × 10⁻⁸ |
| 25922 | Tobramycin | 0.5 | 2.5 × 10⁻⁶ | 1.5 × 10⁻⁷ | 8.7 × 10⁻⁸ |
| | Fosfomycin | 2 | 1.9 × 10⁻⁵ | 6.0 × 10⁻⁶ | 2.5 × 10⁻⁵ |

### Macromolecular Biosynthesis

The effects of antibiotic concentration and time of bacterial exposure to antibiotic on protein and cell wall biosynthesis were determined by measuring the uptake of ³H-aa and ³H-NAG, respectively. Table 4 shows the dose-responses of FT4:1, fosfomycin, and tobramycin. FT4:1 inhibited protein and cell wall biosynthesis to a greater degree than either fosfomycin or tobramycin at 2 h. Increasing the concentration of FT4:1 resulted in increased inhibition of both protein and cell wall biosynthesis; however, protein biosynthesis was inhibited to a greater degree than cell wall biosynthesis. By comparison, increasing the concentration of fosfomycin did not result in increased inhibition of either protein or cell wall biosynthesis.

TABLE 4
Effects of Antibiotic Concentration on Protein and
Cell Wall Synthesis in P. aeruginosa ATCC 27853

% Inhibition^{a}

| Antibiotic (Concentration) | Protein^{b} | Cell Wall^{b} |
|---|---|---|
| FT4:1 (16 μg/mL) | 92 | 59 |
| Fosfomycin (12.8 μg/mL) | 9 | 3 |
| Tobramycin (3.2 μg/mL) | 42 | 36 |
| FT4:1 (8 μg/mL) | 50 | 26 |
| Fosfomycin (6.4 μg/mL) | 21 | 14 |
| Tobramycin (1.6 μg/mL) | 6 | 10 |
| FT4:1 (4 μg/mL) | 28 | 11 |
| Fosfomycin (3.2 μg/mL) | 0 | 8 |
| Tobramycin (0.8 μg/mL) | 2 | 4 |

^{a}% inhibition at 2 h relative to the no-drug control
^{b}Values represent a single experiment

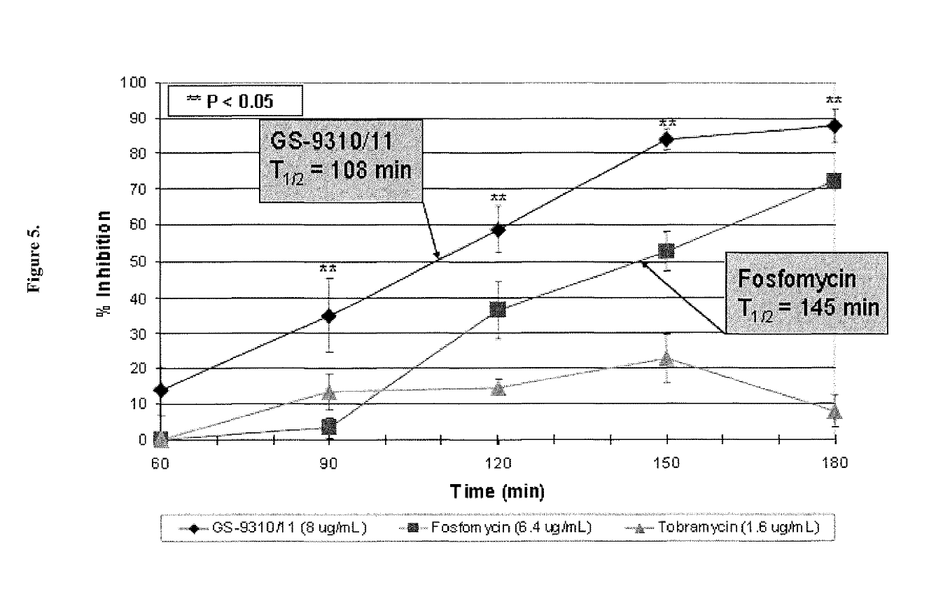

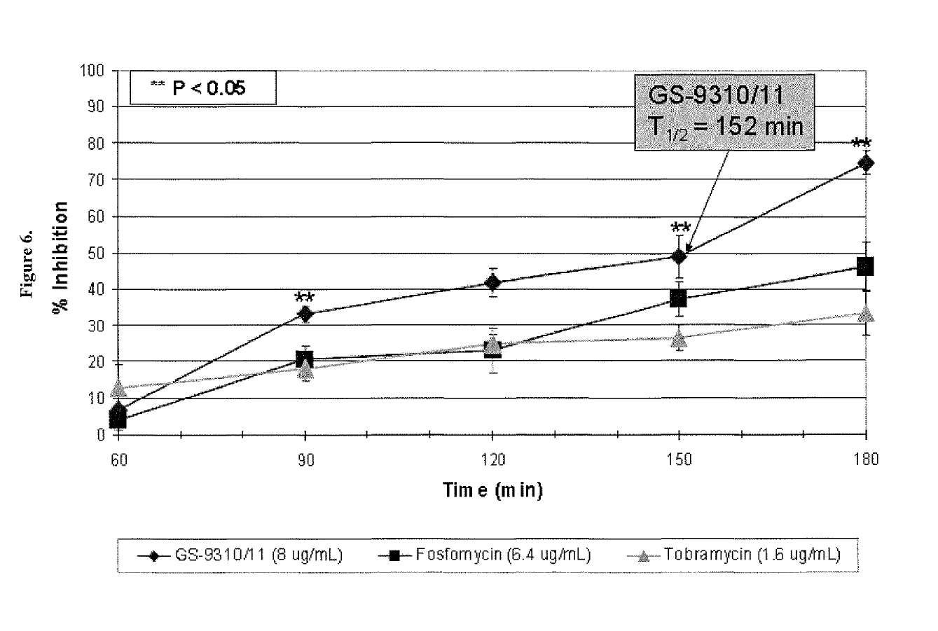

Time-response studies also suggested that FT4:1 was acting primarily through inhibition of protein synthesis. FT4:1 at 8 μg/mL rapidly inhibited 50% (T_{1/2}) of protein synthesis by 108 min compared to 6.4 μg/mL fosfomycin (T_{1/2}=145 min) and 1.6 μg/mL tobramycin (T_{1/2}>180 min) (FIG. 5). In contrast, FT4:1 (8 μg/mL) caused a more gradual inhibition of cell wall biosynthesis (_{T1/2}=152 min), while neither fosfomycin (6.4 μg/mL) nor tobramycin (1.6 μg/mL) reached 50% inhibition within 180 min (FIG. 6).

### Tobramycin Uptake Studies

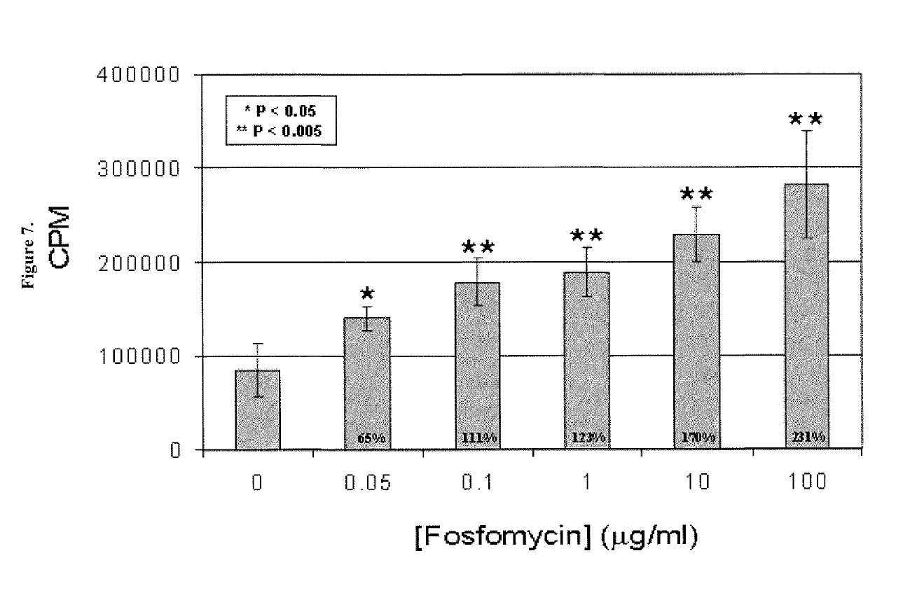

In drug uptake studies, fosfomycin increased bacterial uptake of ³H-tobramycin in a dose-dependent manner; the addition of 10 μg/mL fosfomycin resulted in a 170% increase in ³H-tobramycin uptake relative to the no-fosfomycin control (FIG. 7). These data are complementary to the time-kill experiments that demonstrated that bacterial killing by tobramycin (3.2 μg/mL) was enhanced 3-Log₁₀ CFU/mL in the presence of 12.8 μg/mL of fosfomycin (FIG. 4).

### Discussion

The objective of this study was to address the FT4:1 mechanism of action hypothesis that fosfomycin enhances the uptake of tobramycin into bacterial cells, thereby increasing inhibition of protein synthesis and ultimately, bacterial killing. The major component of FT4:1, fosfomycin, is a phosphonic acid derivative that inhibits cell wall biosynthesis by irreversibly binding to the enzyme UDP-N-acetylglucosamine enoylpyruval transferase (MurA) (Kahan, F M, Ann NY Acad Sci 1974, 235: 364-386). The minor component, tobramycin, is an aminoglycoside that prevents protein biosynthesis by causing translational errors and by inhibiting translocation (Davis, B D, Proc Natl Acad Sci USA 1986; 83: 6164-6168; Tai, P C, Biochem 1979; 18 (1): 193-198). Based on the known mechanisms of action of its components, FT4:1 should act by inhibiting protein and cell wall biosynthesis. However, several lines of evidence suggest the antibacterial activities of the combination are enhanced relative to the individual components. In time-kill experiments conducted in mucin, the activities of FT4:1 for P. aeruginosa were enhanced 500-fold and 1000-fold relative to the components fosfomycin and tobramycin, respectively (MacLeod, D L, Poster 328. 21^{st} Annual North American Cystic Fibrosis Conference, Oct. 3-6, 2007, Anaheim, Calif.) Moreover, the spontaneous mutation frequencies were approximately 700-fold and 170-fold lower for FT4:1 than for either fosfomycin or tobramycin, respectively. Data from time-kill, macromolecular biosynthesis, and drug uptake studies support the proposed FT4:1 mechanism of action hypothesis. Time-kill experiments demonstrated that FT4:1 had killing kinetics similar to tobramycin and that small incremental changes in tobramycin concentration could result in large increases in bacterial killing. Protein and cell wall biosynthesis assays demonstrated that FT4:1 was acting primarily through inhibition of protein biosynthesis. The macromolecular biosynthesis studies also supported time-kill experiments and proved that (i) FT4:1 had enhanced activity relative to the component weights of fosfomycin and tobramycin alone, and (ii) the activities of both FT4:1 and tobramycin were concentration-dependent. Lastly, drug uptake studies demonstrated that fosfomycin increased the accumulation of radiolabeled tobramycin. The exact molecular mechanism accounting for the enhanced activity of FT4:1 is unknown.

The rationale for the FT4:1 combination was to provide a new antibiotic therapy for patients with ophthalmic, otological and dermatological infections that is safe, kills a broad spectrum of bacteria, and has a reduced frequency of resistance relative to monotherapies. FT4:1 is a unique antibiotic combination consisting of a fixed (wt:wt) ratio of fosfomycin and tobramycin. It is active against both gram-negative and gram-positive bacterial pathogens commonly found in ophthalmic, otological and dermatological infections Spontaneous mutation frequencies resulting in antibiotic resistance are also lower with FT4:1 compared to its components, suggesting that it may also be a promising approach to delay the development of resistance in the clinical setting.

### Conclusions

FT4:1 demonstrated enhanced killing relative to its components in a manner that was consistent with the killing kinetics of tobramycin. Macromolecular and antibiotic uptake experiments indicate this was due to fosfomycin increasing the uptake of tobramycin, resulting in increased inhibition of protein biosynthesis and ultimately, bacterial death.

All references cited herein are incorporated by reference in their entireties.

1. A physiologically compatible topical composition for a treatment of a susceptible ophthalmic, otological or dermatological bacterial infection comprising a single dose combination of about 0.001 to about 0.95 mg of fosfomycin, or pharmaceutically acceptable salt thereof, and about 0.001 to about 0.95 mg of tobramycin, or a pharmaceutically acceptable salt thereof, wherein the weight ratio of fosfomycin to tobramycin is from about 5 to about 9 parts fosfomycin to about 1 part to about 5 parts tobramycin.

2. The topical composition of claim 1 wherein the weight ratio of fosfomycin to tobramycin is from about 7 to about 9 parts fosfomycin to from about 1 part to about 3 parts tobramycin.

3. The topical composition of claim 1 wherein the weight ratio of fosfomycin to tobramycin is about 8 parts fosfomycin to about 2 parts tobramycin.

4. The topical composition of claim 1 wherein the single dose combination of fosfomycin and tobramycin comprise about 0.1 to 0.5 percent of the composition.

5. The topical composition of claim 1 wherein the single dose combination comprises less than about 0.3 mg of tobramycin.

6. The topical composition of claim 1 wherein the single dose combination comprises less than about 0.75 mg of the fosfomycin and tobramycin combination.

7. The topical composition of claim 1 wherein the treatment is for a susceptible ophthalmic bacterial infection.

8. The topical composition of claim 1 wherein the treatment is for a susceptible otological bacterial infection.

9. The topical composition of any claim 1 wherein the treatment is for a susceptible dermatological bacterial infection,

10. The topical composition of claim 1 wherein the bacterial infection is selected from the group consisting of S. aureus, S. epidermidis, S. pneumoniae, Pseudomonas aeruginosa, Escherichia coli, Klebsiella pneumoniae, Enterobacter aerogens, Proteus mirabilis, Morganella morganii, Haemophilus influenzae, H. aegyptius, Acinetobacter calcoaceticus and Neissaria species.

11. A physiologically compatible topical composition for treatment of a susceptible ophthalmic, otological or dermatological bacterial infection and inflammation comprising a single dose combination of about 0.001 to about 0.95 mg of fosfomycin, or pharmaceutically acceptable salt thereof, and about 0.001 to about 0.95 mg of tobramycin, or a pharmaceutically acceptable salt thereof, wherein the weight ratio of fosfomycin to tobramycin is from about 5 to about 9 parts fosfomycin to about 1 part to about 5 parts tobramycin, the composition further comprising about 0.001 to about 2 weight percent of at least one anti-inflammatory agent.

12. The topical composition of claim 11 wherein the anti-inflammatory agent is at least one corticosteroid.

13. The topical composition of claim 11 wherein the weight ratio of fosfomycin to tobramycin is from about 7 to about 9 parts fosfomycin to from about 1 part to about 3 parts tobramycin.

14. The topical composition of claim 11 wherein the weight ratio of fosfomycin to tobramycin is about 8 parts fosfomycin to about 2 parts tobramycin.

15. The topical composition of claim 11 wherein the single dose combination of fosfomycin and tobramycin comprise about 0.1 to 0.5 percent of the composition.

16. The topical composition of claim 11 wherein the single dose combination comprises less than about 0.3 mg of tobramycin.

17. The topical composition of claim 11 wherein the single dose combination comprises less than about 0.75 mg of the fosfomycin and tobramycin combination.

18. The topical composition of claim 11 wherein the anti-inflammatory agent is selected from the group consisting of dexamethasone, dexamethasone sodium phosphate, fluorometholone, fluorometholone acetate, loteprednol, loteprednol etabonate, hydrocortisone, prednisolone, fludrocortisones, triamcinolone, triamcinolone acetonide, betamethasone, beclomethasone diproprionate, methylprednisolone, fluocinolone, fluocinolone acetonide, flunisolide, fluocortin-21-butylate, flumethasone, flumetasone pivalate, budesonide, halobetasol propionate, mometasone furoate, fluticasone propionate, ciclesonide, and pharmaceutically acceptable salts thereof.

19. The topical composition of claim 11 wherein the anti-inflammatory agent is selected from the group consisting of dexamethasone, dexamethasone sodium phosphate, fluorometholone, fluorometholone acetate, loteprednol and loteprednol etabonate.

20. The topical composition of claim 11 wherein the treatment is for a susceptible ophthalmic bacterial infection.

21. The topical composition of claim 11 wherein the treatment is for a susceptible otological bacterial infection.

22. The topical composition of claim 11 wherein the treatment is for a susceptible dermatological bacterial infection.

23. The topical composition of claim 11 wherein the bacterial infection is selected from the group consisting of S. aureus, S. epidermidis, S. pneumoniae, Pseudomonas aeruginosa, Escherichia coli, Klehsiella pnewnoniae, Enterobacter aerogens, Proteus mirabilis, Morganella morganii, Haemophilia influenzcie, H. aegyptius, Acinetobacter calcoaceticus and Neissaria species.

24. A method of treating a susceptible ophthalmic, otological or dermatological bacterial infection by administering, to a subject in need thereof, a therapeutically effective amount of a topical composition of claim 1.

25. A use of a topical composition of claim 1 in the manufacture of a medicament for a treatment of a susceptible ophthalmic, otological or dermatological bacterial infection.

26. A method of treating a susceptible ophthalmic, otological or dermatological bacterial infection and inflammation by administering, to a subject in need thereof, a therapeutically effective amount of a topical composition of claim 11.

27. A use of a topical composition of claim 11 in the manufacture of a medicament for a treatment of a susceptible ophthalmic, otological or dermatological bacterial infection and inflammation.

28. (canceled)
